(19) United States
(12) Patent Application Publication
(10) Pub. No.: US 2015/0005740 A1
(43) Pub. Date: Jan. 1, 2015

(54) INJECTION DEVICES AND RELATED METHODS OF USE

(71) Applicant: Boston Scientific Scimed, Inc., Maple Grove, MN (US)

(72) Inventors: Daniel J. FOSTER, Lino Lakes, MN (US); Mary M. BYRON, Roseville, MN (US); James P. ROHL, Prescott, WI (US); Isaac OSTROVSKY, Wellesley, MA (US); Mark W. BODEN, Harrisville, RI (US); Sandra NAGALE, Westford, MA (US); David C. BORZELLECA, Hudson, MA (US); Bradley S. SWEHLA, Eagan, MN (US)

(73) Assignee: Boston Scientific Scimed, Inc., Maple Grove, MN (US)

(21) Appl. No.: 14/318,146

(22) Filed: Jun. 27, 2014

Related U.S. Application Data
(60) Provisional application No. 61/840,866, filed on Jun. 28, 2013.
(60) Provisional application No. 61/889,931, filed on Oct. 11, 2013.

(51) Int. Cl. A61M 5/158

(52) U.S. Cl. CPC A61M 5/158; A61M 2005/1585
USPC 604/506; 604/272; 604/95.01

(57) ABSTRACT

According to an embodiment of the present disclosure, a medical device for injecting one or more substances may include a proximal handle assembly. The proximal handle assembly may include a housing, a rotatable member rotatably coupled to the housing, and a port configured to receive one or more substances. The medical device may also include an inner member coupled to the rotatable member and extending distal to the proximal handle assembly. The medical device may also include an outer member coupled to the housing, surrounding at least a portion of the inner member, and extending distal to the proximal handle assembly. The medical device may also include a helical needle coupled to the inner member and extending distal to the inner member.

### CROSS-REFERENCE TO RELATED APPLICATIONS

This application claims the benefit of priority of U.S. Provisional Application No. 61/840,866, filed Jun. 28, 2013, and U.S. Provisional Application No. 61/889,931, filed Oct. 11, 2013, each of which is incorporated by reference herein in its entirety.

### FIELD OF THE DISCLOSURE

This disclosure relates generally to devices and methods for delivering substances into a subject's body, and in particular, devices and methods for injecting substances into tissue in a subject's body.

### BACKGROUND

Many medical treatments require injecting one or more substances into tissue of a subject's body. One example of a condition that may be treated is overactive bladder (OAB). OAB is a chronic urological condition that may be characterized by pain, urinary frequency, urgency with or without urinary incontinence, and variable degrees of sexual dysfunction. Current treatments for OAB include, for example, medication, diet modification, programs in bladder training, electrical stimulation, and surgery. In some instances, bladder injections can be performed to treat OAB.

With some injection devices, making controlled injections may be difficult. For example, when using straight needles for injections, injection depth may be difficult to monitor and/or control. This may be due to a number of factors, including difficulty gauging the movement distance of straight needles, and deformation of tissue during insertion of straight needles.

Problems with using straight needle injection devices may be exacerbated when conducting medical treatments in which multiple injections may be performed. For example, to treat OAB, multiple injections of substances into bladder tissue may be performed. Ensuring that multiple controlled and consistent bladder injections are being made with a straight needle may be difficult for the reasons set forth above.

Therefore, performance-enhancing injection devices, and related methods of use, are desired.

### SUMMARY

According to an embodiment of the present disclosure, a medical device for injecting one or more substances may include a proximal handle assembly. The proximal handle assembly may include a housing, a rotatable member rotatably coupled to the housing, and a port configured to receive one or more substances. The medical device may also include an inner member coupled to the rotatable member and extending distal to the proximal handle assembly. The medical device may also include an outer member coupled to the housing, surrounding at least a portion of the inner member, and extending distal to the proximal handle assembly. The medical device may also include a helical needle coupled to the inner member and extending distal to the inner member.

According to another embodiment of the present disclosure, a medical device for injecting one or more substances may include a catheter having a proximal end and a distal end. The distal end of the catheter may include a distal end wall extending substantially perpendicular to a longitudinal axis of the catheter. The medical device may also include a curved needle having a proximal end and a distal end. The proximal end of the curved needle may be coupled to the distal end wall of the catheter, adjacent an edge of the distal end wall of the catheter.

According to another embodiment of the present disclosure, a method for treating a patient's bladder with a medical device may include positioning a proximal handle assembly of the medical device outside of the patient's body. The proximal handle assembly may include a housing, a rotatable member rotatably coupled to the housing, and a port configured to receive one or more substances. The method may also include inserting at least a portion of an inner member of the medical device, coupled to the rotatable member and extending distal to the proximal handle assembly, into the patient's body, and at least a portion of an outer member of the medical device into the patient's body, the outer member being coupled to the housing, surrounding at least a portion of the inner member, and extending distal to the proximal handle assembly. The method may also include positioning a helical needle of the medical device, coupled to the inner member and extending distal to the inner member, in the patient's bladder adjacent bladder tissue. The method may also include rotating the rotatable member to rotate the inner member and the helical needle, and move the helical needle distally with a needle drive mechanism. The method may also include penetrating the bladder tissue with the helical needle. The method may also include inserting the helical needle into the bladder tissue to a desired depth. The method may also include injecting the one or more substances into the bladder tissue at the predetermined depth.

According to an embodiment of the present disclosure, a medical device for injecting a substance may include a rotatable tube coupled to a distal helical needle. The medical device may also include a drive assembly coupled to the rotatable tube. The drive assembly may be configured to rotate the rotatable tube. The medical device may also include an actuation member coupled to the drive assembly. The actuation member may be configured to move between a proximal position and a distal position, to actuate the drive assembly to rotate the rotatable tube. The medical device may also include a connector on the rotatable tube configured to receive a source of the substance.

According to another embodiment of the present disclosure, a medical device for injecting a substance may include a rotatable tube including a distal helical needle. The medical device may also include a drive assembly coupled to the rotatable tube. The drive assembly may be configured to rotate the rotatable tube. The medical device may also include an actuation member coupled to the drive assembly. The actuation member may be configured to actuate the drive assembly to rotate the rotatable tube when moving between a first position and a second position. The medical device may also include at least one biasing member coupled to the actuation member. The at least one biasing member may be configured to position the actuation member in an intermediate position between the first position and the second position.

According to another embodiment of the present disclosure, a method for injecting a substance into tissue with a medical device may include engaging a surface of the tissue with a helical needle of the medical device. The method may also include rotating the helical needle in a tissue penetrating direction to penetrate the tissue. Rotating the helical needle in the tissue penetrating direction may include moving an actuation member from a rest position to a first position, to rotate the helical needle through a first angle of rotation in the tissue penetrating direction. The method may also include injecting the substance into the tissue through the helical needle. The method may also include rotating the helical needle in a disengaging direction opposite the tissue penetrating direction to withdraw the helical needle from the tissue.

Additional characteristics and advantages of the disclosure will be set forth in part in the description, which follows, and in part will be apparent from the description, or may be learned by practice of the disclosure.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain principles of the disclosure.

FIG. 1 is a schematic view of a urinary tract.

FIGS. 2A and 2B are side views of an exemplary medical device, according to principles of the present disclosure.

FIGS. 3A and 3B are perspective views of a portion of a medical device, according to principles of the present disclosure.

FIG. 4 is a side view of a needle assembly, according to principles of the present disclosure.

FIG. 5 is a side view of a portion of a medical device, according to principles of the present disclosure.

FIGS. 6A-6C are side views of a portion of a medical device, according to principles of the present disclosure.

FIG. 7 is a side view of a portion of a medical device, according to principles of the present disclosure.

FIG. 8A is a perspective view of a needle, according to principles of the present disclosure.

FIG. 8B is a schematic view of exemplary dimensional characteristics of the needle of FIG. 8A, according to principles of the present disclosure.

FIG. 8C is a perspective view of a needle, according to principles of the present disclosure.

FIG. 9 is a side view of an exemplary medical device, according to principles of the present disclosure.

FIG. 10A is a close-up view of a proximal portion of the exemplary medical device of FIG. 9, according to principles of the present disclosure, with some components shown in partially transparent form to more clearly depict relationships between components.

FIG. 10B is a close-up side view of a fluid connector, according to principles of the present disclosure.

FIG. 10C is a close-up front view of a pin vise, according to principles of the present disclosure.

### DESCRIPTION OF THE EMBODIMENTS

Reference is now made in detail to embodiments of the present disclosure, examples of which are illustrated in the accompanying drawings. Wherever possible, the same reference numbers will be used throughout the drawings to refer to the same or like parts.

### Overview

The present disclosure is generally directed to a medical device that can inject one or more substances to predetermined depths, at multiple sites, in a region of tissue. Embodiments of the medical device may include a curved needle configured to penetrate bladder wall tissue. However, use of the medical device is not limited to the bladder or the urinary tract, and embodiments may be applied to deliver substances to any organ, lumen, cavity, and/or other target area of a subject's body.

The disclosed medical devices and methods are provided for exemplary purposes, and are not intended as limiting the scope of the present disclosure.

### Exemplary Embodiments

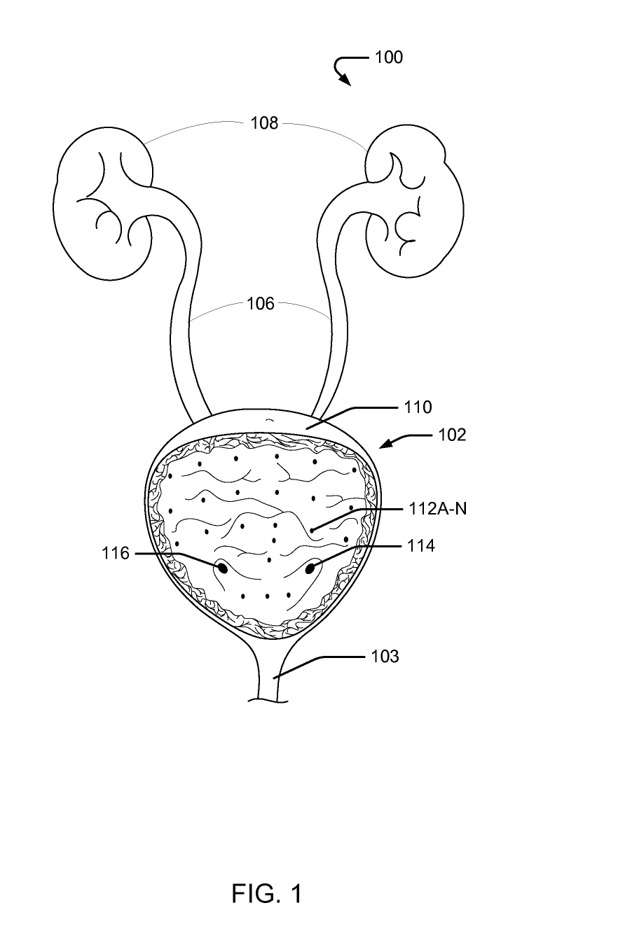

FIG. 1 is a schematic view of a urinary tract 100. The urinary tract 100 may include a bladder 102, a urethra 103, kidneys 108, and ureters 106. The bladder 102 may have openings 114 and 116 for the right and left ureters 106. A wall of the bladder 102 is identified by reference numeral 110.

One or more substances may be injected into the wall 110 of the bladder 102 to treat OAB. For example, the injections may be performed at injection sites 112A-N. It is contemplated that 15-50 injection sites may be appropriate for a majority of subjects. However, the number of injections, the locations of the injections, the depths of the injections, and the amounts/types of substances injected, may vary based on a subject's anatomy, the type of procedure being performed, and/or based on other factors. As an example, for a subject with a large bladder, more injections may be used than for a subject with a smaller bladder. For subjects with thicker bladder walls, deeper injections may be used, and/or larger amounts of substances may be injected, than for subjects with thinner bladder walls.

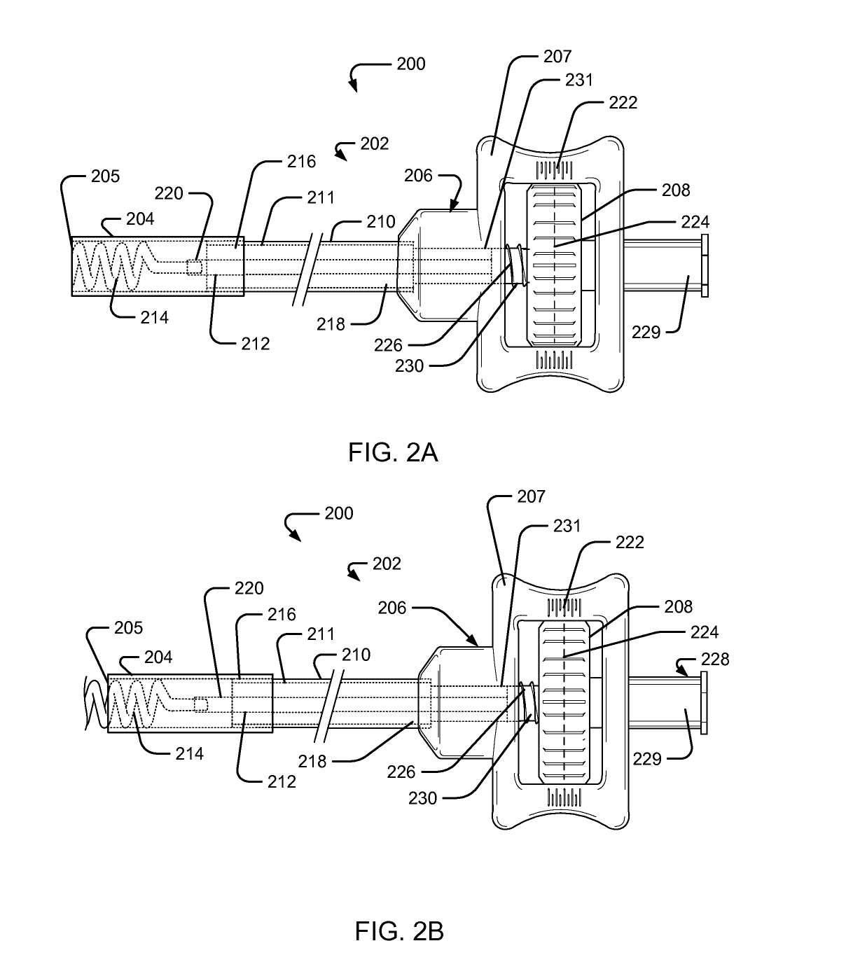

FIGS. 2A and 2B show an exemplary medical device 200 for injecting substances (not shown) into the wall 110 of the bladder 102 (FIG. 1). The medical device 200 may include an insertion unit 202, and a proximal handle assembly 206. The insertion unit 202 may be inserted into a subject's body, and may be navigated to a target area therein. The proximal handle assembly 206 may remain outside of the subject's body, where it may be manipulated by a user.

The insertion unit 202 may include an outer member 210, an inner member 212, a needle 214, and a needle housing 204. The outer member 210 may be tubular, and may have a proximal portion 218 and a distal portion 216. The inner member 212 may be tubular, and may be configured to pass through a lumen formed by the inner surface 211 of the outer member 210. At least a portion of the inner member 212 may extend distal to the distal portion 216 of the outer member 210. The outer member 210 and the inner member 212 may be made of flexible hypodermic tubing. The tubing may be metallic, ceramic, polymeric, made of natural substances, or any combination of these materials. The outer and inner members 210 and 212 may have any suitable dimensions, and that their dimensions may vary based on the type of treatment being performed and/or a subject's anatomy.

The needle 214 may have a spiral/corkscrew/helical shape. The needle 214 may be attached to a distal region 220 of the inner member 212. The needle 214 may be formed of stainless steel, or any other suitable materials. The dimensions of the needle 214 may vary depending on the type of treatment being performed, and/or the anatomy of the subject. The needle 214 may have a flat grind or a multifaceted grind with bevels, at its distal end, to help the needle 214 penetrate tissue. The interior of the needle 214 may be covered with a hydrophobic coating. The needle 214 may have any suitable cross-sectional profile (e.g., circular, elliptical, rectangular, or square), and/or any combination of cross-sectional profiles, along the length of the needle 214. For example, the needle 214 may have a distal portion with a rectangular cross-sectional profile, and a proximal portion with a circular cross-sectional profile.

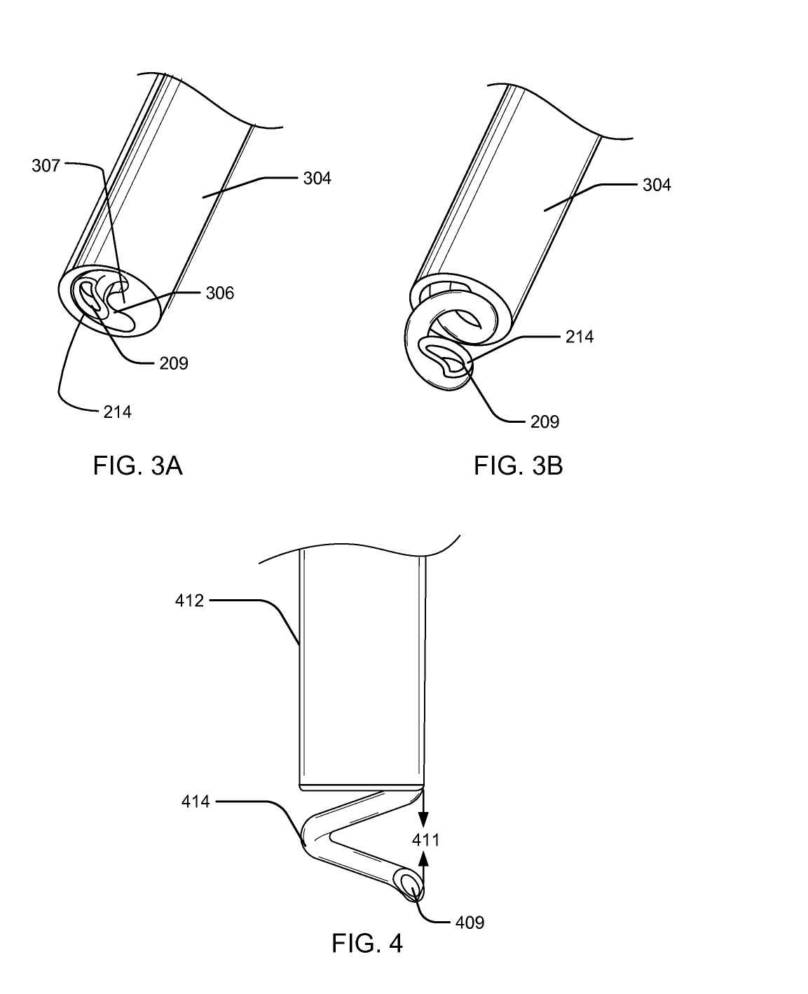

The needle housing 204 may surround at least a portion of the needle 214, the outer member 210, and the inner member 212. A distal end of the needle housing 204 may have an opening 205 therein through which the needle 214 can pass. As shown in FIG. 2A, the needle 214 may be positioned within the needle housing 204 in a first, retracted state. This may help prevent contact between the needle 213 and tissue in a subject's body during insertion of the needle 214 into the subject's body, guiding of the needle 214 to the target area, and withdrawal of the needle 214 from the target area and/or the subject's body after the procedure has been performed. As shown in FIG. 3A, the needle 214 may be extended out from within the needle housing 204 to a second, extended state, so the exposed portion of the needle 214 may penetrate tissue at the target area. It is contemplated that the needle housing 204 may be transparent, and/or may include ruler markings (not shown) along its length, for monitoring and measuring movement of the needle 214 relative to the needle housing 204 using an imaging device (not shown) capable of providing images of the needle 214 and needle housing 204 to the user.

The proximal handle assembly 206 may include a housing 207 coupled to the proximal portion 218 of the outer member 210. The proximal handle assembly 206 may be made of one or more polymers and/or metals, or any other suitable substances. A knob 208 may be rotatably coupled to the housing 207. The knob 208 may be substantially cylindrical, and side portions of the knob 208 may be accessible to the user on each side of the housing 207. The knob 208 may include a shaft 230, with external threads 226. The shaft 230 may be coupled to a proximal end of the inner member 212. The threads 226 may engage complementary threads in a recess 231 in the housing 207. As the user rotates the knob 208 in a first direction (e.g., one of clockwise or counterclockwise), engagement between the threads may cause distal movement of the knob 208, inner member 212, and needle 214. As the user rotates the knob 208 in a second direction, opposite the first direction, engagement between the threads may cause proximal movement of the inner member 212 and needle 214. It is contemplated that in one embodiment, the complementary threads may be removed, and another mechanism may be used to convert rotation of the knob 208 into proximal and distal movement of the needle 214.

The housing 207 may include a gauge 222, and the knob 208 may include a marking 224. By monitoring relative movement between the marking 224 and the gauge 222, the user may be provided with an indication of how far the knob 208 has moved relative to the housing 207. Because the needle 214 is coupled to the knob 208 by the inner member 212, and the needle housing 204 is coupled to the housing 207 by the outer member 210, movement of the knob 208 relative to the housing 207 corresponds to movement of the needle 214 relative to the needle housing 204. Thus, the marking 224 and the gauge 222 also provide an indication of how far the needle 214 has moved relative to the distal end of the needle housing 204. If the distal end of the needle housing 204 rests on a surface of tissue in the target area, the marking 224 and the gauge 222 may provide an indication of how far the needle 214 has penetrated the tissue.

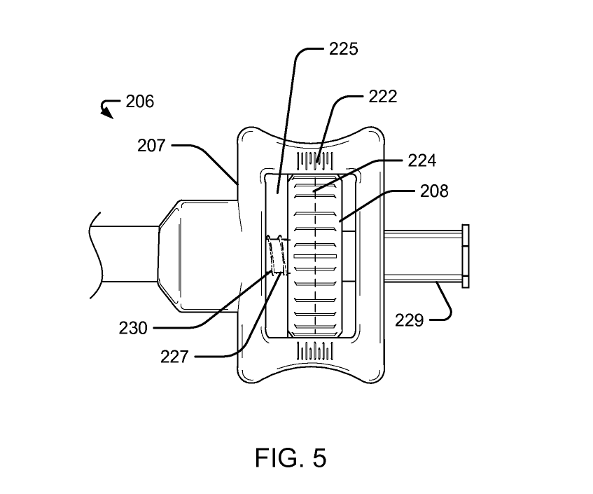

It is also contemplated that the proximal handle assembly 206 may include a hard stop for limiting movement of knob 208, and thus limiting movement of the needle 214 relative to the distal end of the needle housing 204. The hard stop may include, for example, a clip 225 (FIG. 5) having a central opening 227. The clip 225 may be removably inserted into the housing 207, with the central passage 227 removably receiving the shaft 230. The clip 225 may engage opposing surfaces of the housing 207 and the knob 208, thereby limiting distal travel of the knob 208 relative to the housing 207, and also distal travel of the needle 214 relative to the needle housing 204. Clips having different thickness may also be provided so the user can adjust the limit on distal travel based on a subject's anatomy and/or the type of procedure being performed. One or more clips, similar to the clip 225, may be provided for positioning on the proximal side of the knob 208, to limit proximal travel of the needle 214 relative to the needle housing 204.

The proximal handle assembly 206 may further include a port 229 with a proximal end that may be fluidly coupled to a source (not shown), such as a container, reservoir, canister, syringe, pipette, and/or any other device suitable for storing and dispensing one or more substances. The port 229 may also be coupled to the knob 208. A fluid passage (not shown) may extend through the port 229, knob 208, shaft 230, inner member 212, and needle 214. It is contemplated that the proximal end 228 may include a luer lock mechanism or other suitable connection for coupling the port 229 and the source. The source may include any suitable substances dispenser, including those with a mechanism that provides a user the ability to set and dispense predetermined volumes of substances in a consistent and repeatable manner. Substances supplied by the source may include, for example, Botox, hydrogels, polymers/copolymers like PEG (polyethylene glycol) or pluronics, anticholinergics, combinations of these substances, and/or any other suitable substances.

FIGS. 3A and 3B show a needle housing 304 having an opening 306 at the distal end or tip of the housing 304. The needle 214 may be extendable out of, and retractable into, the housing 304, via the opening 306. The opening 306 may be C-shaped, with its contours partially formed by a protrusion 307. As the needle 214 is rotated, sloped outer surface portions of the needle 214 may engage one or more edges of the housing 304 that form a periphery of the opening 306. The engagement causes the needle 214 to extend out of, and retract into, the housing 304, along a longitudinal axis of the housing 304, when the needle 214 is rotated. Thus, the needle 214 and the opening 306 may provide a mechanism for needle movement. FIG. 3B shows the needle 214 after it has been rotated and extended out of the housing 304, so the needle 214 may be used to penetrate tissue, and deliver substances into the tissue via a needle lumen/opening 209. The edges forming the periphery of the opening 306, and/or the protrusion 307, may help stabilize the needle 214 during insertion of the needle 214 into tissue, by engaging outer surfaces of the needle 214, and providing additional structural support. Rotation of the needle 214 in an opposite direction may cause the needle 214 to retract into the housing 304, so that the housing 304 can prevent the needle 214 from inadvertently engaging and damaging tissue when injections are not being performed.

In the embodiment shown in FIG. 4, a needle 414 may be coupled to a distal end of a member 412. A distal surface of the member 412 may act as a stop. For example, when the needle 414 is inserted into tissue, once the distal surface of the member 412 engages the surface of the tissue, further penetration of the needle 414 into the tissue may be prevented. If the needle 414 is used with the housing 304, once the distal surface of the member 412 engages the distal end of the housing 304, further penetration of the needle 414 into the tissue may be prevented, due to engagement between the member 412 and portions of the housing 304 around the opening 306. Thus, a height of the curved needle 414, shown as reference number 411 (FIG. 4), may correspond to the depth of insertion of the needle 414 into the tissue. It is contemplated that in some embodiments, the needle 414 and the member 412 may be used in place of the needle 214 and inner member 212.

The dimensions of the needle 414 may vary based on a number of factors. One of those factors is the substance being injected using the needle 414. For example, the needle 414 may be a 22 gauge needle if hydrogel is injected using the needle 414, and the needle 414 may be a 25 gauge needle if Botox is injected using the needle 414. The needle height 411 may be 1-3 mm for hydrogel delivery, and 3-5 mm for Botox delivery. It is also contemplated that the member 412 may have a diameter of 1.5 mm or 2.5 mm, depending on the type of substance being injected. It is also contemplated that the member 412 may have an outer diameter of 0.020″ and an inner diameter of 0.016″, an outer diameter of 0.018″ and an inner diameter of 0.016″, or an outer diameter of 0.018″ and an inner diameter of 0.0176″. Additional factors that may influence the dimensions of the needle 414 may include the type of procedure being performed, and anatomical characteristics of a subject. The needle 414 may have any suitable cross-sectional profile (e.g., circular, elliptical, rectangular, or square), and/or any combination of cross-sectional profiles, along the length of the needle 414. For example, the needle 414 may have a distal portion with a rectangular cross-sectional profile, and a proximal portion with a circular cross-sectional profile.

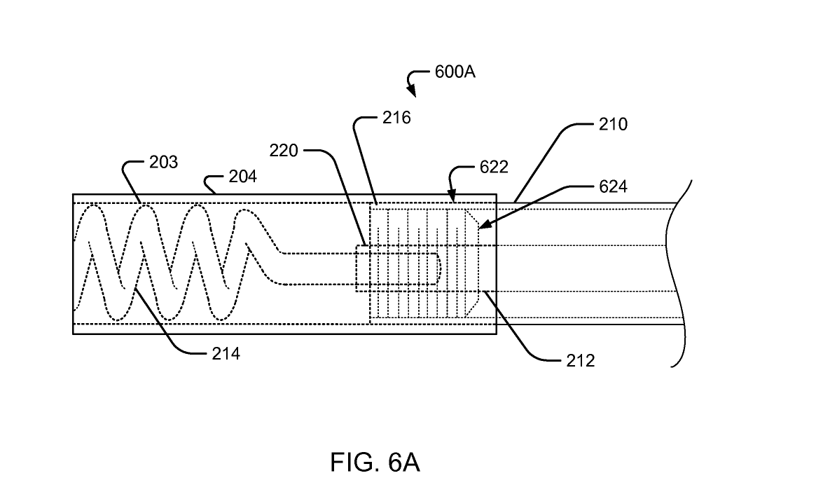

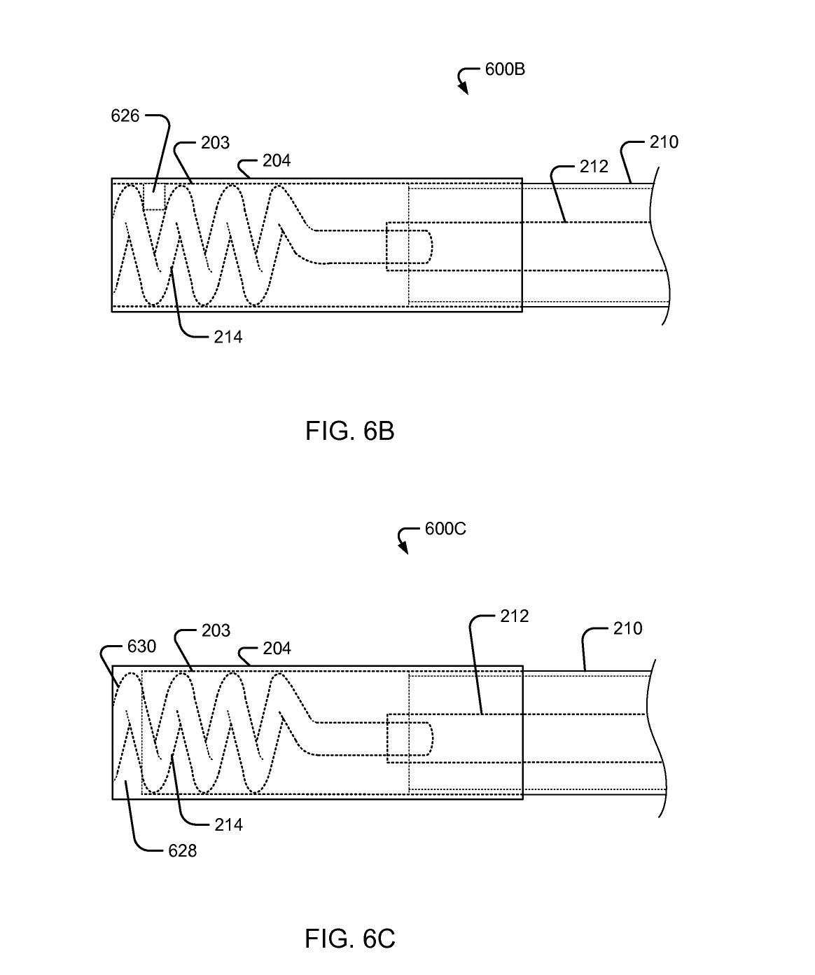

FIGS. 6A-6C show assemblies 600A, 600B, and 600C, respectively, each assembly including a mechanism for providing needle movement. FIG. 6A shows the needle 214 retracted into the needle housing 204. In this embodiment, a threaded male coupler 624 may be attached to a distal portion of the inner member 212. The male coupler 624 may include, for example, threads similar to those on a screw. The threaded male coupler 624 may engage a complementary female threaded coupler 622 at a distal portion 216 of the outer member 210. For example, the female threaded coupler 622 may include a threaded portion on an interior surface of the outer member 210.

If the inner member 212 and the threaded male coupler 624 are rotated in a first direction (e.g., one of clockwise or counterclockwise), engagement between the threads of the threaded male coupler 624 and the female threaded coupler 622 may cause distal movement of the inner member 212 and the needle 214. If the user rotates the inner member 212 and the threaded male coupler 624 in a second direction, opposite the first direction, engagement between the threads of the threaded male coupler 624 and the female threaded coupler 622 may cause proximal movement of the inner member 212 and the needle 214.

In the embodiment of FIG. 6B, the needle housing 204 may include a protrusion or post 626 on its internal surface 203. The post 626 may engage sloped outer surface portions of the needle 214. As the needle 214 is rotated in a first direction, the sloped outer surface portions may engage the post 626. The engagement may cause extension of the needle 214 from the housing 204, along a longitudinal axis of the housing 204. Rotation of the needle 214 in a second direction, opposite the first direction, may cause retraction of the needle 214 into the housing 204.

In the embodiment of FIG. 6C, the needle housing 204 may include an end wall 628 including a spiral/corkscrew/helix shaped opening 630. Surfaces of the opening 630 may engage sloped outer surface portions of the needle 214. As the needle 214 is rotated in a first direction, the sloped outer surface portions may engage the contours of the opening 630. The engagement may cause extension of the needle 214 from the housing 204, along a longitudinal axis of the housing 204. Rotation of the needle 214 in a second direction, opposite the first direction, may cause retraction of the needle 214 into the housing 204. Further, because a straight proximal portion of the needle 214 may not fit in the curved opening 630, the proximal portion of the needle 214 and the opening 630 may together form a stop to limit distal travel of the needle 214 relative to the needle housing 204.

It is contemplated that the device 200 (FIGS. 2A and 2B) may include one or more of the features shown in FIGS. 3A, 3B, and 6A-6C. For example, the needle housing 204 may include the distal opening 306 and protrusion 307. In such an embodiment, engagement between the needle 214 and the edges of the housing 204 around the opening 306, during rotation of the needle 214 using the knob 208, may provide one mechanism for moving the needle 214 along the longitudinal axis of the housing 204. Engagement between the threads 226 and the recess 231 may provide another mechanism for moving the needle 214 (proximally or distally) along the longitudinal axis of the housing 204. Additionally or alternatively, the inner member 212 may include the threaded male coupler 624, and the outer member 210 may include the female threaded coupler 622, to provide another mechanism for moving the needle 214. Additionally or alternatively, the housing 204 may include the post 626 to provide another mechanism for moving the needle 214. Additionally or alternatively, the housing 204 may include the end wall 628 and opening 630 to provide another mechanism for moving the needle 214. While any combination of the above-described mechanisms for moving the needle 214 along the longitudinal axis of the housing 204 may be provided, it is also contemplated that only one of the mechanisms may be provided to simplify construction of the device 200.

It is also contemplated that the device 200 may not include the engaged threaded portions 226 and 231, distal opening 306, protrusion 307, threaded male coupler 624, female threaded coupler 622, post 626, end wall 628, or opening 630, for moving the needle 214. Rather, according an aspect of the disclosure, in one exemplary embodiment, the needle 214 may be rotated without being driven longitudinally by any of the aforementioned needle drive mechanisms. When the needle 214 engages/penetrates tissue, rotation of the needle 214 in a first direction, in combination with the helical shape of the needle 214, may cause the needle 214 to penetrate further into the tissue in the manner of a corkscrew. Thus, the rotating needle 214 may act as its own needle drive mechanism, drawing itself deeper into the tissue via its rotation, without requiring a separate needle drive mechanism. Rotation of the needle 214 in the second direction may back the needle 214 out of the tissue.

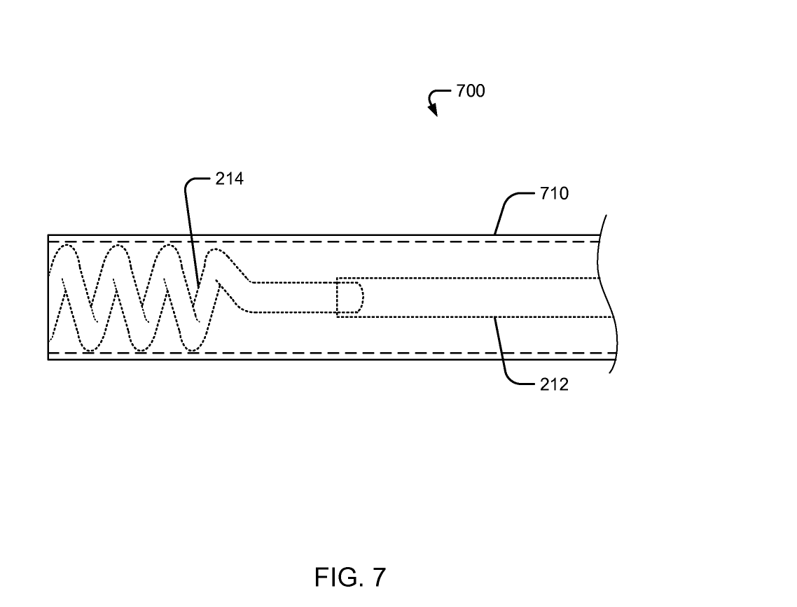

FIG. 7 shows an embodiment where a distal portion of an outer member 710, similar to the outer member 210, provides a housing for the helical needle 214. This arrangement eliminates the need for a separate needle housing. Features of any of the needle movement mechanisms shown in FIGS. 2A, 2B, 3A, 3B, and 6A-6C, may be used in conjunction with the embodiment of FIG. 7, either alone or in combination, with the distal portion of the outer member 706 in place of the housing 204 or the housing 304. Alternatively, the embodiment of FIG. 7 may be constructed without the needle movement mechanisms, and rotation of the needle 214 against the tissue may draw the needle 214 into and/or retract the needle 214 out of the tissue.

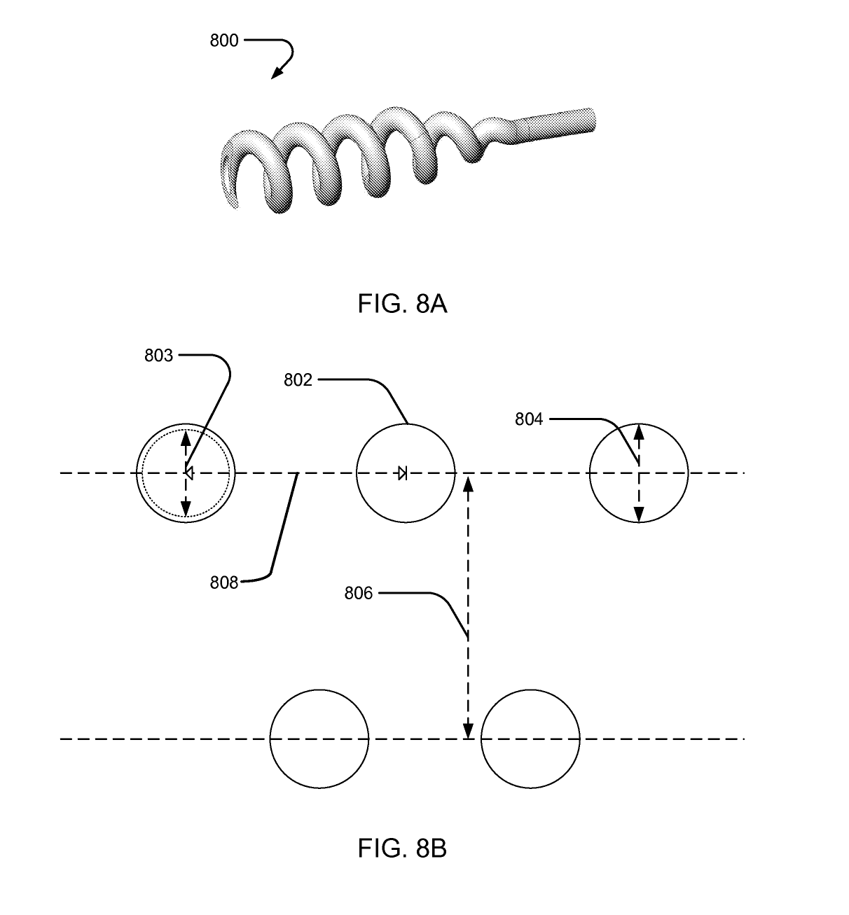

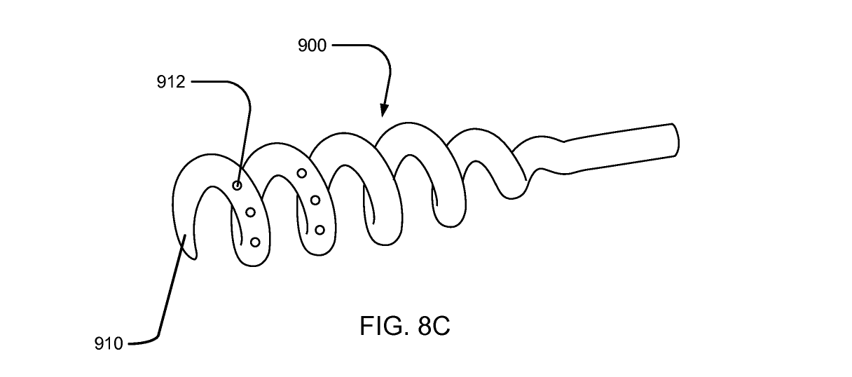

FIG. 8A is a perspective view of an exemplary curved needle 800. FIG. 8B is a schematic illustrating various dimensional characteristics of the needle 800. Reference numeral 802 is associated with a cross-section of a portion of the needle 800. As shown, the needle 800 may have an inner diameter 803, an outer diameter 804, and a centerline diameter 806. In one embodiment, the needle 800 may be a 23 gauge needle with an outer diameter of 0.0160″, an outer diameter to inner diameter ratio of 1.8, and an outer diameter to centerline diameter ratio of 3.4. In another embodiment, the needle 800 may be a 20 gauge needle with an outer diameter of 0.0358″, an outer diameter to inner diameter ratio of 3, and an outer diameter to centerline diameter ratio of 5.25. In another embodiment, the needle 800 may be a 32 gauge needle with an outer diameter of 0.0093″, an outer diameter to inner diameter ratio of 1.3, and an outer diameter to centerline diameter ratio of 1.20.

The needle 800 may also have a pitch 808. The pitch 808 may be a width of one complete turn of the helix of the needle 800, measured parallel to a longitudinal axis of the needle 800. It is contemplated that the needle 800 may have a single consistent pitch. For example, a distal portion of the needle 800 may have the same pitch as a proximal portion of the needle 800. Alternatively, the needle 800 may have a variable pitch. For example, a distal portion of the needle 800 may have a larger or smaller pitch than a proximal portion of the needle 800. Needle movements and depth of penetration may be controlled by selecting one or more pitches for the needle 800. For a needle portion with a first pitch, rotation of the needle portion through a first angle may result in a first depth of penetration of the needle portion into the tissue. For a needle portion with a second pitch larger than the first pitch, rotation of the needle portion through the first angle may result in a second depth of penetration of the needle portion into the tissue, the second depth of penetration being larger than the first depth of penetration.

It is also contemplated that the needle 800 may have a single consistent centerline diameter 806. For example, a distal portion of the needle 800 may have the same centerline diameter as a proximal portion of the needle 800. Alternatively, the needle 800 may have a variable centerline diameter. For example, a distal portion of the needle 800 may have a larger or smaller centerline diameter than a proximal portion of the needle 800. As a more detailed example, a distal portion of the needle 800 may have a larger centerline diameter than a proximal portion of the needle 800, with the centerline diameter tapering down from the distal end to the proximal end of the needle 800. The distal portion of the needle 800 may follow a broader diameter path than the proximal portion during insertion/rotation of the needle 800. Needle movements and depth of penetration may be controlled by selecting one or more centerline diameters for the needle 800.

It is also contemplated that needle 800 may be flexible, and may have a constrained configuration, with a first centerline diameter, when being delivered to a target area. A constraining force may be exerted by a needle housing, similar to the needle housing 204, needle housing 304, or outer member 710. Constraining the needle 800 may make delivery easier due, for example, to the smaller width of the needle 800, and the smaller dimensions of the needle housing required to hold the constrained needle 800. As the needle 800 exits from the needle housing, the needle 800 may expand to an unconstrained configuration, with a second centerline diameter larger than the first centerline diameter.

It is also contemplated that the needle 800 may have variable stiffness along its length due to the use of different materials in different portions of the needle 800, and/or due to variations in the pitch and/or centerline diameter of the needle 800. It is also contemplated that the needle 800 may have a straight segment, instead of being curved throughout.

The needle 214, or the needle 414, may have any of the above-listed dimensions, pitches, centerline diameters, and/or characteristics of the needle 800.

According to another aspect of the present disclosure, a needle 900 may include a closed distal tip 910, and a plurality of side ports 912 that allow fluid to exit from the needle 900 (FIG. 8C). It is contemplated that the needle 214, needle 414, and/or needle 800 may have the closed distal tip and side ports of the needle 900.

The disclosed medical device may provide enhanced or proper depth control, and may facilitate or ensure that the substance is injected at the desired depth within the tissue, such as a bladder wall. In addition, the disclosed medical device may include any mechanism for fixing the needle before injection, and for providing the ability to control the injection volume and/or inject multiple aliquots. The steerable properties of the disclosed medical device may result in reduced maneuvering and enhanced or improved ergonomics for a physician or other caregiver performing injections at multiple locations, which may potentially result in less pain for the patient.

The disclosed embodiments and their components may be manufactured using a variety of manufacturing methods. Examples may include, but are not limited to, stamping, press rolling, soldering, brazing, molding, and the like. Further, suitable substances employed to manufacture the device may include any suitable biocompatible substances, such as metals including stainless steel, aluminum, titanium, polymers, composites, and the like. However, the above manufacturing methods and substances are merely provided for exemplary purposes, and are not intended to limit the scope of the present disclosure.

Exemplary method steps associated with the above-described embodiments will now be described. A first step may include navigating a needle, such as the needle 214 or the needle 414, to a target area of a subject's body. This step may include inserting the insertion unit 202 of the medical device 200 into the subject's body, with the needle in a retracted position within the needle housing 204, needle housing 304, or outer member 710.

It is also contemplated that a catheter, endoscope, or similar insertion device (not shown) may be inserted into the subject's body and navigated to the target area. As known in the art, the insertion device may include a steering mechanism to help deflect distal portions of the insertion device, and help the insertion device navigate into and through the subject's body. For example, the insertion device may include a plurality of longitudinally extending cables or wires positioned around a circumference of the insertion device. The wires may be coupled to a proximal handle, including one or more knobs. Rotation of the one or more knobs may pull certain wires, and/or push certain wires, to steer/deflect the distal portion of the insertion device.

The insertion device may also include one or more lumens for receiving one or more instruments. For example, the insertion device may include a lumen for receiving an imaging device, such as a camera or sensor, allowing the imaging device to be positioned at or adjacent a distal end of the insertion device. The imaging device allows the user to visualize the target area. An exemplary insertion device (a video endoscope), is described in U.S. Pat. No. 7,578,786 B2 to Boulais et al., titled “Video Endoscope,” which was issued Aug. 25, 2009, and its contents are herein incorporated by reference. It is also contemplated that the insertion device may also include a lumen for another instrument. This other instrument may be configured to change a state of the injected substance. The other instrument may include, for example, a heated wire or laser, configured to crosslink or solidify the injected substance. Additionally or alternatively, the needle itself may be heated to bring about the change of state. Additionally or alternatively, fluids that solidify upon contact to form a new desired material, may be introduced via different lumens. Examples of such fluids are collagen and glutaraldehyde, and also cross-linked hydrogels. It is also contemplated that the needle may have multiple parallel lumens extending therethrough, for separating the fluids, and then allowing them to mix when exiting from the distal end of the needle.

The insertion unit 202 may be inserted through a lumen of the insertion device after the insertion device has reached the target area, or the insertion unit 202 may already be positioned within the insertion device during movement of the insertion device to the target area, such that deflection of the insertion device by the steering mechanism may also cause a similar deflection of the insertion unit 202. A distal end of the insertion unit 202 may be positioned at, adjacent to, or distally beyond the distal end of the insertion device. It is also contemplated that the distal end of the insertion unit 202 may be visualized by the imaging device. The proximal handle assembly 206 of the medical device 200 may remain outside of the subject's body, where it can be manipulated by the user.

The needle may be placed adjacent and/or against tissue at the target area. The target area may be the subject's bladder 102, and the tissue may be the tissue forming the bladder wall 110. Placing the needle adjacent and/or against the tissue may include abutting the tissue with a distal end of the needle housing 204, needle housing 304, or outer member 710. The user may accurately place the needle using the imaging device to visually guide the needle to the target area, and using the insertion device steering mechanism to position the needle.

With the needle in position, penetration of the tissue with the needle may begin. Penetrating the tissue with the needle may include rotating the knob 208 in a first direction. As the knob 208 is rotated, the needle may rotate and move distally, thus piercing the tissue, and penetrating deeper into the tissue as the knob 208 continues to rotate. Rotation of the knob 208 in the first direction may lead to distal movement of the needle due to engagement between complementary threads on the shaft 230 and the recess 231, the needle surface and edges surrounding the opening 306, complementary threads on the threaded male coupler 624 and the female threaded coupler 622, the post 626 and the needle surface, and/or the opening 630 of the wall 628 and the needle surface. Alternatively, rotation of the knob 208 in the first direction may lead to distal movement of the needle due to engagement between the helical needle surface and the tissue, with the needle acting as its own needle drive mechanism, and the engaged threaded portions 226 and 231, distal opening 306, protrusion 307, threaded male coupler 624, female threaded coupler 622, post 626, end wall 628, and opening 630 may be omitted.

When the needle has reached a desired depth, the user may stop rotating the knob 208. The desired depth may be the depth at which the distal opening at the distal tip of the needle has reached a layer of tissue forming part of the wall 110, or between layers forming part of the wall 110. The user may position the needle at the desired depth by: monitoring movement of the marking 224 relative to the gauge 222, and discontinuing rotation of the knob 208 when a desired distance between the marking 224 and the gauge 222 has been reached; discontinuing rotation of the knob 208 when the distal end of the inner member 412 contacts the tissue or the edges surrounding the opening 306, the post 626, and/or the wall 628; discontinuing rotation of the knob 208 when a straight portion of the needle abuts against the edges surrounding the opening 306, and/or the wall 628; rotating the knob 208 until its movement is stopped by the clip 225; and/or rotating the knob 208 until the needle has moved a desired distance as measured by ruler markings (not shown) on a transparent portion of the distal end portion of the needle housing 204, 304, or 710 that receives at least a portion of the needle.

With the needle at the desired depth, the user may deliver one or more substances into the port 229. The substance may be injected in predetermined quantities. The substance may flow through the passage in the port 229, knob 208, shaft 230, inner member 212 or inner member 412, and needle 214 or 414, into the tissue.

After injecting the substance, the user may withdraw the needle from the tissue. Withdrawal may include rotating the knob 208 in a second direction, opposite the first direction. The same needle drive mechanisms that cause extension of the needle when the knob 208 is rotated in the first direction, may cause retraction of the needle when the knob 208 is rotated in the second direction. The needle may be retracted until the needle is once again enclosed within the needle housing 204, needle housing 304, or outer member 710.

The user may know when to stop rotating the knob 208 in the second direction by: monitoring movement of the marking 224 relative to the gauge 222, and discontinuing rotation of the knob 208 when a desired distance between the marking 224 and the gauge 222 has been reached; rotating the knob 208 until its movement is stopped by a clip (not shown), similar to clip 225, positioned on a proximal side of the knob 208; and/or rotating the knob 208 until the needle has moved a desired distance as measured by ruler markings on the transparent portion of the distal end portion of the needle housing 204, 304, or 710 that receives at least a portion of the needle.

The user may reposition the needle adjacent and/or against tissue at another site in the target area. For example, the user may move the insertion device using the steering mechanism, to move the needle. The user may use the imaging device to ensure the needle is moved to the desired site. Once there, the user may repeat the penetration, injection, and withdrawal steps listed above. It is contemplated that, for treatment of OAB, these steps may be repeated between 15 and 50 times to inject substances into 15 to 50 sites on the bladder wall 110. When the desired number of injections has been made, insertion unit 202 of the medical device 200 may be withdrawn from the subject's body, with the needle in the retracted position.

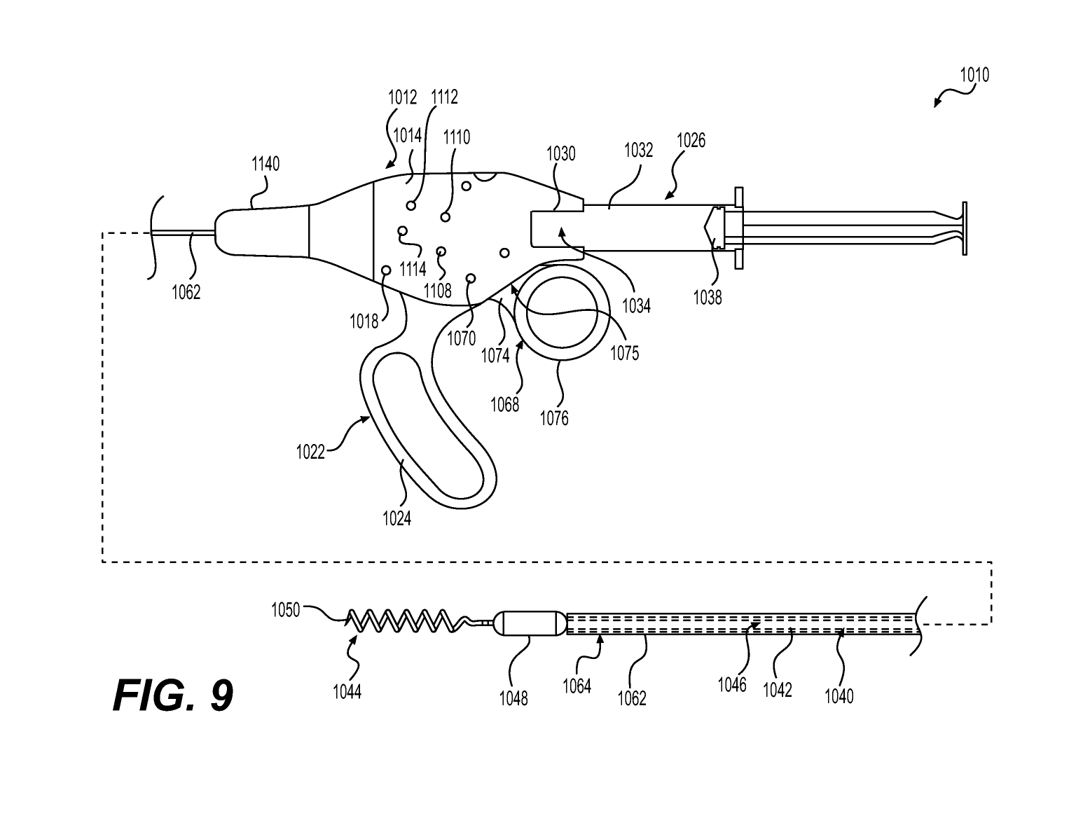

FIG. 9 shows an exemplary medical device 1010 for injecting a substance (not shown), such as one or more fluids, into tissue of a subject's body. The medical device 1010 may include a proximal housing 1012. The housing 1012 may include two half-shells 1014, only one of which is visible in FIG. 9. The half-shells 1014 may be joined by any suitable fastening mechanism 1018, such as one or more screws, rivets, and/or adhesives. Additionally or alternatively, the half-shells 1014 may be heat-sealed to one another along their contacting edges, which may run longitudinally along the center of the housing 1012. Additionally or alternatively, the half-shells 1014 may be joined by friction fit, and/or by snap-fit engagement. When the half-shells 1014 are joined together, interior surfaces of the half-shells 1014 may define one or more passages 1020 extending longitudinally through the housing 1012. In some embodiments, the housing 1012 can separate into more than two sections. In some embodiments, the housing 1012 may be a single structure sized and shaped to accommodate actuator components.

The housing 1012 may include a grip member 1022. For example, each of the half-shells 1014 may include half of the grip member 1022. Alternatively, one of the half-shells 1014 may include the entire grip member 1022. The grip member 1022 may remain stationary with respect to the other portions of the housing 1012 when the medical device 1010 is in use. The grip member 1022 may include a loop 1024 through which one or more of a user's fingers may be inserted.

The housing 1012 may be formed of any suitable material, including one or more injection moldable polymers, for example acrylonitrile butadiene styrene (ABS), Nylon or polyethylene. Polymers may include reinforcement materials such as glass fibers. Additionally or alternatively, the housing 1012 may be metallic, and may include stainless steel, aluminum, and/or any other suitable metals or metal alloys.

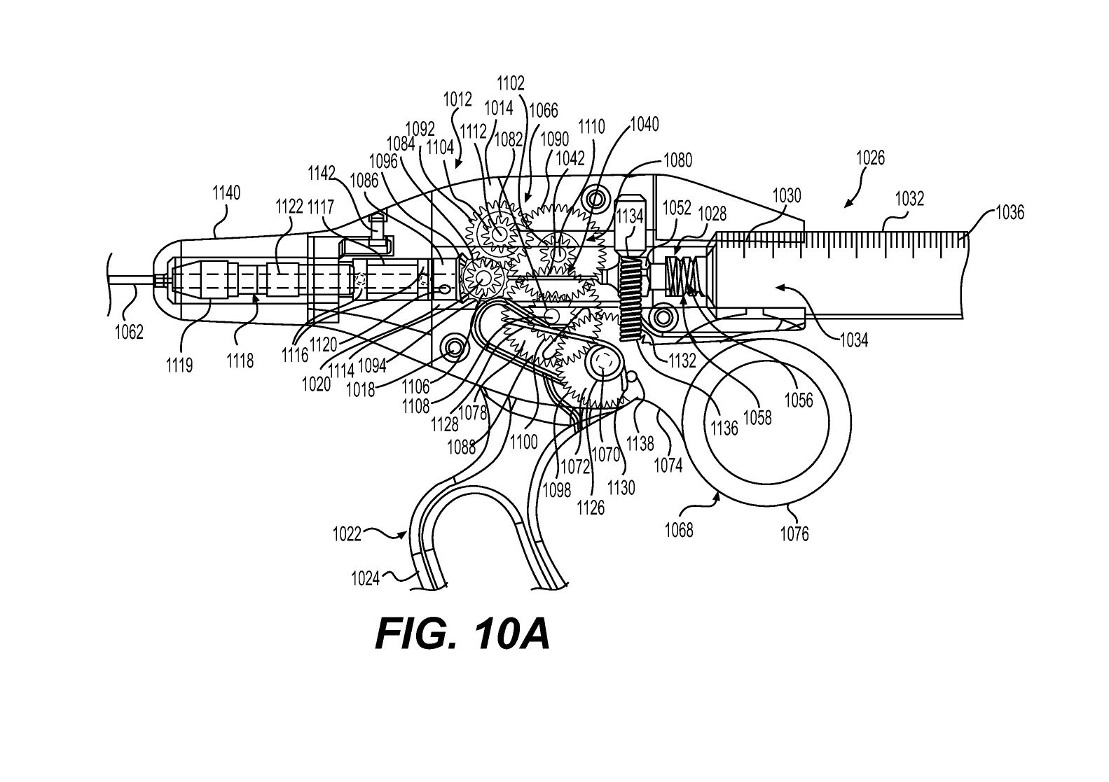

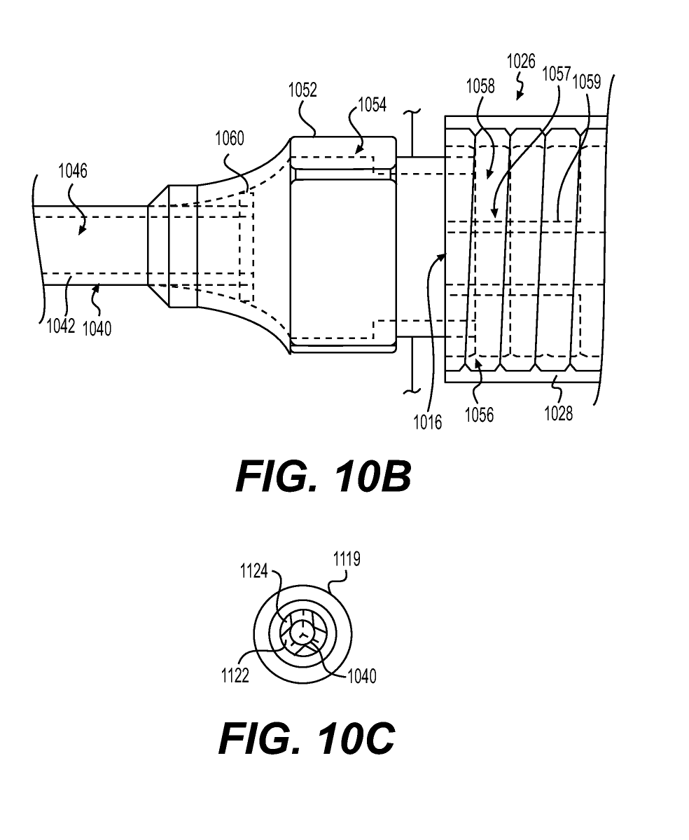

The medical device 1010 may also include a syringe 1026. The proximal end of the housing 1012 may include a chamber 1028 (FIG. 10A) configured to receive the distal end of the syringe 1026, and a cavity 1030 configured to receive a tubular body portion 1032 of the syringe 1026. One or more openings 1034 in the portion of the housing 1012 forming the cavity 1030 may allow viewing of the tubular body portion 1032 when the syringe 1026 is inserted into the housing 1012, and in particular, viewing of the fluid contained within the interior of the tubular body portion 1032. The one or more openings 1034 may also allow viewing of measurement markings 1036 on the outer surface of the tubular body portion 1032.

A plunger 1038 may be slidably received in the proximal end of the tubular body portion 1032. When the plunger 1038 is forced in the distal direction by a user, the plunger may push the fluid in the tubular body portion 1032 out of a distal opening 1016 in the distal end of the syringe 1026.

It is contemplated that the syringe 1026 may be replaced by any other suitable source of fluid. For example, the syringe 1026 may be replaced by a conduit (not shown) connected to a pump and/or reservoir containing the fluid. It is also contemplated that the syringe 1026 may be replaced by a pressurized fluid canister.

The medical device 1010 may also include a hypotube 1040 (FIGS. 9 and 10A). The hypotube 1040 may include a proximal shaft 1042 and a distal helical needle 1044. A lumen 1046 (FIG. 9) may extend through the hypotube 1040, with portions of the lumen 1046 extending through the shaft 1042 and the helical needle 1044. The hypotube 1040 may be made of flexible hypodermic tubing, composed of any suitable metallic, polymeric, or biocompatible material, or any combination of these materials. For example, the hypotube 1040 may be made of Nitinol and/or stainless steel. It is contemplated that, in one embodiment, the shaft 1042 may be made of Nitinol, while the helical needle 1044 may be made of stainless steel. Using different materials for the different regions of the hypotube 1040 may give the hypotube 1040 more flexibility in one region, and more rigidity in another region. The needle 1044 may have any cross-sectional shape and/or size, and this shape and/or size may vary along the length of the needle 1044.

The shaft 1042 and the helical needle 1044 may be coupled by any suitable attachment mechanism 1048. For example, the shaft 1042 and the helical needle 1044 may be coupled by swaging, welding, crimping, and/or joining using an epoxy. It is also contemplated that the shaft 1042 and the helical needle 1044 may be coupled by friction fit.

The helical needle 1044 may include a distal opening 1050 at its distal end, and one or more of the helical needle features shown in, for example, FIGS. 8A-8C. Even so, in some embodiments the needle 1044 may include side apertures for infusing or aspirating fluids, similar to those shown in FIG. 8C. In some embodiments, the distal end of the needle 1044 may be closed (FIG. 8C).

As shown in FIGS. 10A and 10B, the proximal end of the shaft 1042 may be coupled to the distal end of the syringe 1026 by a connector 1052. A passage 1054 (FIG. 10B) may extend between the proximal and distal ends of the connector. The proximal end of the connector 1052 may be coupled to the distal end of the syringe 1026. For example, the distal end of the syringe 1026 may include internal threads 1056 configured to engage a flange 1058 on the proximal end of the connector 1052. The flange 1058 may include external threads complementary to the threads 1056. Rotating the flange 1058 relative to the threads 1056 may screw one of the connector 1052 and the syringe 1026 onto the other of the connector 1052 and the syringe 1026. The distal end of the syringe 1026 may also include a fitting or tubular portion 1059 configured to be received in an opening 1057 in the flange 1058 in a fluid-tight manner. The passage 1054 of the connector 1052 may be in fluid communication with the distal opening 1016 of the syringe 1026 so that the fluid may flow out of the syringe 1026 and through the connector 1052. It is also contemplated that the connector 1052 and the syringe 1026 may be coupled by any suitable luer lock connection. In some embodiments, the connector and syringe may be connected with a snap fit which operates simply by pushing the two components together until they fit into one another, for example by having a protrusion (e.g., a ridge or bump) snap into a corresponding groove.

The distal end of the connector 1052 may be coupled to the proximal end of the hypotube 1040. The passage 1054 through the connector 1052 may be in fluid communication with the lumen 1046 of the hypotube 1040. The fluid may flow through the connector 1052 and into the lumen 1046.

It is contemplated that the proximal end of the hypotube 1040 may be fixedly coupled to the distalmost end of the connector 1052. For example, the proximal end of the hypotube 1040 may be glued, welded, crimped, or swaged to the distal end of the connector 1052. In such an embodiment, the hypotube 1040, connector 1052, and syringe 1026 may move as a unit.

Alternatively, the proximal end of the hypotube 1040 may extend into the passage 1054 of the connector 1052 (FIG. 10B), and a seal 1060 may be provided between the hypotube 1040 and the connector 1052. The seal 1060 may include, for example, an o-ring configured to engage the inner surface of the connector 1052 and the outer surface of the hypotube 1040, to reduce or eliminate leakage of fluid from between the connector 1052 and the hypotube 1040.

The o-ring may have an irregularly shaped cross-section, such as an X-shaped, U-shaped, or C-shaped cross-section. The o-ring may have a radial cavity on a proximally facing surface, and one or more radial lips beside the radial cavity for engaging the external surface of the hypotube 1040 and the internal surface of the connector 1052. Increasing fluid pressure in the passage 1054 of the connector 1052 may force the lips radially outward, thus increasing the sealing force between the lips and at least one of the hypotube 1040 and the connector 1052.

Alternatively, the seal 1060 may include a first sealing ring (not shown) coupled to the hypotube 1040 and a second sealing ring (not shown) coupled to the connector 1052, wherein opposing axial surfaces of the first and second sealing members engage to form the seal. Increasing fluid pressure in the passage 1054 of the connector 1052 may force the first and second sealing rings into tighter engagement, thus enhancing sealing.

In embodiments including the seal 1060, the hypotube 1040 may be rotated relative to the connector 1052. For example, the connector 1052 and the syringe 1026 may be locked in place relative to the housing 1012, by friction fit or any suitable attachment mechanism, while the hypotube 1040 may rotate relative to the housing 1012, connector 1052, and syringe 1026.

The medical device 1010 may also include an outer sheath 1062. The outer sheath 1062 may include a lumen 1064 (FIG. 9) extending between its proximal and distal ends. The proximal end of the outer sheath 1062 may be coupled to the distal end of the housing 1012. The shaft 1042 of the hypotube 1040 may extend through the lumen 1064. The distal helical needle 1044 may extend distally out from the distal end of the outer sheath 1062. The outer sheath 1062 may be metallic, polymeric, biocompatible, or any combination of such materials. In some embodiments, the outer sheath 1062 may be reinforced, for example, by metallic or polymeric braiding.

It is contemplated that an additional outer sheath (not shown) may be sized to slide over the hypotube 1040 and the outer sheath 1062. The additional outer sheath may include a central lumen through which the hypotube 1040 and the outer sheath 1062 may be inserted. The additional outer sheath may protect tissue from the helical needle 1044 as the helical needle 1044 is inserted to a target site. Additionally or alternatively, a stylet (not shown) may be inserted through the helical needle 1044 to straighten the helical needle 1044 when the helical needle 1044 is inserted to a target site. Once the straightened helical needle 1044 reaches the target site, the stylet may be removed, allowing the helical needle 1044 to move back to its curved configuration.

The medical device 1010 may also include a drive assembly 1066 (FIG. 10A). The drive assembly 1066 may be received in the one or more passages 1020 in the interior of the housing 1012. The drive assembly 1066 may include an actuation member 1068. The actuation member 1068 may be movably coupled to the housing 1012, and may move relative to the housing 1012 between a proximal position and a distal position. The proximal position may be a proximalmost position, where further proximal movement of the actuation member 1068 may be prevented by the actuation member 1068 coming into contact with a surface of the housing 1012. The distal position may be a distalmost position where further distal movement of the actuation member 1068 may be prevented by the actuation member 1068 coming into contact with a surface of the housing 1012. Alternatively, the proximal and distal positions may be between travel limits of the actuation member 1068.

In one embodiment, the actuation member 1068 may be configured to pivot about a rotational axis 1070, relative to the housing 1012. An axis engaging portion 1072 (FIG. 10A) of the actuation member 1068 may be housed within the one or more passages 1020 in the housing 1012. An arm portion 1074 of the actuation member 1068 may extend out from the one or more passages 1020 via a slot or opening 1075 (FIG. 9) in the housing 1012, and away from the housing 1012. A ring portion 1076 of the actuation member 1068 may be coupled to the arm portion 1074.

In use, the user may insert his or her fingers into the loop 1024 of the grip member 1022, and his or her thumb into the ring portion 1076. By moving his or her thumb towards his or her fingers, the user may move the actuation member 1068 toward the second position. By moving his or her thumb away from his or her fingers, the user may move the actuation member 1068 toward the first position.

The drive assembly 1066 may also include one or more drive members 1078, 1080, 1082, 1084, and 1086 (FIG. 10A). The outer periphery of each of the drive members 1078, 1080, 1082, and 1084 may include one or more drive elements 1088, 1090, 1092, and 1094. The drive members 1078, 1080, 1082, and 1084 may also include one or more driven elements 1100, 1102, 1104, 1106 radially inward of the drive elements 1088, 1090, 1092, and 1094. The drive member 1086 may include one or more driven elements 1096.

The outer periphery of the axis engaging portion 1072 may include one or more drive elements 1098 configured to engage the one or more driven elements 1100 of the first drive member 1078. One or more drive elements 1088 of the first drive member 1078 may engage one or more driven elements 1102 of the second drive member 1080, one or more drive elements 1090 of the second drive member 1080 may engage one or more driven elements 1104 of the third drive member 1082, one or more drive elements 1092 of the third drive member 1082 may engage one or more driven elements 1106 of the fourth drive member 1084, and one or more drive elements 1094 of the fourth drive member 1084 may engage one or more driven elements 1096 of the fifth drive member 1086.

In one embodiment, the drive members 1078, 1080, 1082, and 1084 may include gear wheels configured to rotate about rotational axes 1108, 1110, 1112, and 1114, respectively. The drive member 1086 may include a bevel gear configured to rotate about the longitudinal axis of the hypotube 1040. The axis engaging portion 1072 may include a partial gear wheel, and the one or more drive/driven elements 1088, 1090, 1092, 1094, 1096, 1098, 1100, 1102, 1104, and 1106 may include gear teeth. The rotational axes 1070, 1108, 1110, 1112, and 1114 may include pins extending through the housing 1012, the pins being supported on their respective ends by openings in the half-shells 1014. Alternatively, the rotational axes 1070, 1108, 1110, 1112, and 1114 may include one or more axle-shaped protrusions formed on an interior surface of the housing 1012. The axes of rotation of each of the actuation member 1068 and the drive members 1078, 1080, 1082, and 1084, may be substantially perpendicular to the longitudinal axis of the hypotube 1040. The axis of rotation of the drive member 1086 may be substantially coaxial with the longitudinal axis of the hypotube 1040.

Movement of the actuation member 1068 may cause movement of the first drive member 1078, which may cause movement of the second drive member 1080, which may cause movement of the third drive member 1082, which may cause movement of the fourth drive member 1084, which may cause movement of the fourth drive member 1084 may cause movement of the fifth drive member 1086. For example, in one embodiment, pivoting the axis engaging portion 1072 clockwise toward the distal position may cause counterclockwise rotation of the first drive member 1078, which may cause clockwise rotation of the second drive member 1080, which may cause counterclockwise rotation of the third drive member 1082, which may cause clockwise rotation of the fourth drive member 1084, which may cause clockwise rotation of the fifth drive member 1086. Pivoting the axis engaging portion 1072 counterclockwise toward the proximal position may reverse the rotations of the first, second, third, fourth, and fifth drive members 1078, 1080, 1082, 1084, and 1086. In some embodiments, there may be fewer drive members depending on the amount and type of control needed for the helical needle 1044. In some embodiments, there may be more drive members depending on the amount and type of control needed for the helical needle 1044. In some embodiments, one or more of the drive members may be made with a transmission or with a locking mechanism for selecting one or more preferred drive members for use, while bypassing or locking other drive members. With such an arrangement, control of the helical needle 1044 may be adjusted using the transmission or locking mechanism without requiring opening of the housing 1012.

In another embodiment, the actuation member 1068 may be replaced by a dial (not shown) rotatably coupled to the housing 1012. The dial may be an enlarged version of the axis engaging portion 1072, with at least a portion of the dial extending out of the housing 1012. A user may manually rotate the dial to rotate the axis engaging portion 1072, and thereby actuate the drive members 1078, 1080, 1082, 1084, and 1086. In some embodiments, one or more of the drive members 1078, 1080, 1082, 1084, and 1086 may include a dial rotatably coupled to the housing 1012 and accessible from outside of the housing 1012. The dial may be manually rotated by a user to actuate one or more of the drive members 1078, 1080, 1082, 1084, and 1086.

A pin vise 1118 may be coupled to the fifth drive member 1086. For example, a proximal portion 1117 of the pin vise 1118 may be received in a cavity (not shown) of the fifth drive member 1086, and a set screw 1120 may be used to secure the proximal portion 1117 in the cavity. The proximal portion 1117 may be supported within one or more bearing assemblies 1116. Each bearing assembly 1116 may include, for example, an annular ring including an outer surface for engaging the housing 1012, and an inner surface for engaging the proximal portion 1117, to facilitate rotation of the pin vise 1118 relative to the housing 1012.

The fifth drive member 1086 and pin vise 1118 may move as a unit. For example, the fifth drive member 1086 and pin vise 1118 may rotate together. It is also contemplated that the fifth drive member 1086 may be removable from the pin vise 1118 by tightening or loosening a set screw 1120.

A lumen 1122 may extend through the pin vise 1118 and fifth drive member 1086. The lumen 1122 may receive the hypotube 1040. For example, the shaft 1042 of the hypotube 1040 may extend through the lumen 1122.

The pin vise 1118 may include one or more clamping members 1124 (FIG. 10C) configured to selectively move radially inward to engage the hypotube 1040, thus fixing the hypotube 1040 to the pin vise 1118, and move radially outwardly to disengage from the hypotube 1040, thus releasing the hypotube 1040 from the pin vise 1118. The clamping members 1124 may be evenly spaced circumferentially around the hypotube 1040. Thus, when the clamping members 1124 engage the hypotube 1040, clamping forces exerted on the hypotube 1040 may be evenly distributed around the circumference of the hypotube 1040. It is contemplated that rotation of the proximal portion of the pin vise 1118 relative to the rest of the pin vise 1118, in a first direction, may move the clamping members 1124 radially inward, and rotation of the proximal portion in a second direction, opposite the first direction, may move the clamping members 1124 radially outward. The pin vise 1118 may be similar to those used to hold drills.

When the pin vise 1118 closes around the hypotube 1040, the hypotube 1040 may be fixed to the pin vise 1118. The hypotube 1040 and the pin vise 1118 may include matching components, such as a protrusion (e.g., a bump) on one and a recess (e.g., a divot) on the other, or a square receiving section of the pin vise 1118 and a square profile of a proximal end of the hypotube 1040. Thus, the hypotube 1040 may rotate together with the pin vise 1118 and fifth drive member 1086, and may rotate with the pin vise 1118 and fifth drive member 1086 relative to the bearing assembly 1116. The bearing assembly 1116 may help ensure that the hypotube 1040, pin vise 1118, and fifth drive member 1086 remain centered during rotation. When the pin vise 1118 opens, it may release from the hypotube 1040. This may allow for removal of the hypotube 1040 from the housing 1012. The selective opening and closing of the pin vise 1118 may provide for efficient interchanging of one hypotube for another. Access to the pin vise 1118 may be provided by removing a proximal portion 1140 of the housing 1012 to expose the pin vise 1118. The proximal portion 1140 may be coupled to the rest of the housing 1012 by, for example, one or more attachment mechanisms 1142. The one or more attachment mechanisms 1142 may include, for example, one or more set screws. In some embodiments, the hypotube 1040 may be connected to the fifth drive member 1086 by additional or alternative connection mechanisms, such as screw fittings, friction fittings, clamps, and/or other suitable connection mechanisms. The connection mechanisms may replace or augment the pin vise 1118.

Based on characteristics of the drive assembly 1066, movement of the actuation member 1068 through a distance may produce a number of rotations of the hypotube 1040. This measure may be referred to as a distance to rotation ratio. Also, based on those characteristics, movement of the actuation member through the distance may require exertion of a force on the actuation member by the user. For example, the shape, number, dimensions, and relative positions of the axis engaging portion 1072 and the drive members 1078, 1080, 1082, 1084, and 1086, may affect the distance to rotation ratio and/or the required force. The shape, number, dimensions, and relative positions of the drive/driven elements 1088, 1090, 1092, 1094, 1096, 1098, 1100, 1102, 1104, and 1106, may also affect the distance to rotation ratio and/or the required force.

A first combination of the above-described characteristics may result in a first distance to rotation ratio, and a first required force. Based on the type of treatment being performed, a subject's anatomy, and/or user preferences, the user may want to change the distance to rotation ratio and/or the required force. To make adjustments, the user may open the housing 1012 by, for example, separating the half-shells 1014. The user may then modify the drive assembly 1066. For example, the user may replace one of the drive members 1078, 1080, 1082, 1084, and 1086, with a drive member having one or more different characteristics, designed to produce a predetermined desired distance to rotation ratio and/or required force. More than one of the drive members 1078, 1080, 1082, 1084, and 1086 may be replaced, if desired. Additionally or alternatively, the actuation member 1068 may be replaced by another actuation member with one or more different characteristics. The drive members 1078, 1080, 1082, and 1084, and the actuation member 1068, may slide off of their respective rotational axes 1070, 1108, 1110, 1112, and 1114, and replacement parts may slide onto the rotational axes 1070, 1108, 1110, 1112, and 1114. The drive member 1086 may be removed by loosening a locking member such as a set screw 1120, to free the drive member 1086 from the bearing assembly 1116, and a replacement part may be added by tightening its associated set screw once it is on the bearing assembly 1116. In some embodiments, one or more of the drive members may be made with a transmission or with a locking mechanism for selecting one or more preferred drive members for use, while bypassing or locking other drive members. With such an arrangement, control of the helical needle 1044 may be adjusted using the transmission or locking mechanism without requiring opening of the housing 1012.

The drive assembly 1066 may also include a biasing member 1126. The biasing member 1126 may include a torsion spring. A first leg 1128 of the biasing member 1126 may engage the housing 1012, and a second leg 1130 of the biasing member 1126 may engage the actuation member 1068. For example, the second leg 1130 may engage a protrusion 1138 on the actuation member 1068. The biasing member 1126 may bias the actuation member 1068 toward the proximal position shown in FIG. 10A.

The drive assembly 1066 may also include a biasing member 1132. The biasing member 1132 may include a coil spring. A first end 1134 of the biasing member 1132 may engage the housing 1012, and a second end 1136 of the biasing member 1132 may engage the actuation member 1068. The biasing member 1132 may bias the actuation member 1068 toward the distal position.

In embodiments where both biasing members 1126 and 1132 are present, the biasing forces of the biasing members 1126 and 1132 may act against each other. An equilibrium point may be established where the biasing force exerted by the biasing member 1126 on the actuation member 1068, is substantially equal to the biasing force exerted by the biasing member 1132 on the actuation member 1068. A rest position (FIG. 10A) for the actuation member 1068 can be found at the equilibrium point. The rest position may be an intermediate position between the proximal and distal positions of the actuation member 1068.

When the actuation member 1068 is proximal to the intermediate position, the biasing force of the biasing member 1132 may be greater than the biasing force of the biasing member 1126, and thus, the actuation member 1068 may be moved distally to the intermediate position by the resultant biasing force, in the absence of a force exerted on the actuation member 1068 by the user. When the actuation member is distal to the intermediate position, the biasing force of the biasing member 1126 may be greater than the biasing force of the biasing member 1132, and thus, the actuation member 1068 may be moved proximally to the intermediate position by the resultant biasing force, in the absence of a force exerted on the actuation member 1068 by the user.

When the user moves the actuation member 1068 from the intermediate position toward the distal position a first distance, the hypotube 1040 may rotate through a first angle, causing the helical needle 1044 to penetrate into tissue to a first depth. To withdraw the helical needle 1044 from the tissue, the user may move the actuation member 1068 back to the intermediate position, or allow such movement at the urging of the biasing member 1126. However, in some instances, a portion of the helical needle 1044 may remain within the tissue. This may be because the tissue, when experiencing compressive forces during insertion of the helical needle 1044, may deform a first amount. The same tissue, when experiencing tensile forces during removal of the helical needle 1044, may deform a second amount greater than the first amount. In other words, in some instances, the rotation angle for removing the helical needle 1044 from tissue, may be greater than the rotation angle for inserting the helical needle 1044 into the tissue. In some embodiments, a portion of the device 1010, for example the housing 1012, may include one or more markings to allow the user to correlate the movement of the helical needle 1044 to the movement of the actuation member 1068.

By positioning the actuation member 1068 in the intermediate position, the biasing members 1126 and 1132 may provide the additional movement distance needed to withdraw the helical needle 1044 after it has been inserted into tissue. Returning to the example above, after the actuation member 1068 has reached the intermediate position during withdrawal, the helical needle 1044 will have undergone rotation in the withdrawal/disengaging direction through an angle equal to the angle of rotation undergone in the insertion direction. Because at least a portion of the helical needle 1044 may remain in the tissue, additional rotation of the hypotube 1040 may be needed to fully withdraw the helical needle 1044 from the tissue. The additional rotation may be provided by moving the actuation member 1068 from the intermediate position to the proximal position.

The extent of the additional rotation needed to remove the helical needle 1044 from the tissue may depend, for example, on the subject's anatomy. The biasing forces exerted by the biasing members 1126 and 1132 may be adjusted to provide a predetermined or desired amount of additional rotation. For example, if an increased amount of additional rotation is needed, the biasing member 1132 may be replaced with a biasing member having a greater biasing force, and/or the biasing member 1126 may be replaced with a biasing member having a lesser biasing force, so that the intermediate position of the actuation member moves distally. If, on the other hand, less additional rotation is needed, the biasing member 1126 may be replaced with a biasing member having a greater biasing force, and/or the biasing member 1132 may be replaced with a biasing member having a lesser biasing force, so that the intermediate position of the actuation member moves proximally.

In another embodiment, only one of the biasing members 1126 and 1132 may be present. For example, only the biasing member 1126 may be present. In such an embodiment, the intermediate position may be the position of the actuation member 1068 at which the proximal biasing force exerted by the biasing member 1126 ceases to force the actuation member 1068 in the proximal direction due, for example, to inertia associated with the drive assembly 1066. The user may still rotate the actuation member 1068 in the proximal direction, which may result in the protrusion 1138 coming out of contact with the second leg 1130. Alternatively, only the biasing member 1132 may be present. In such an embodiment, the intermediate position may be the position of the actuation member 1068 at which the distal biasing force exerted by the biasing member 1132 ceases to force the actuation member 1068 in the distal direction due, for example, to inertia associated with the drive assembly 1066. The user may rotate the actuation member 1068 in the distal direction, which may result in the actuation member 1068 separating from the biasing member 1132.

Additionally or alternatively, the intermediate position may correspond to a position where the protrusion 1138 engages a complementary structure (not shown) on an inner surface of the housing 1012. For example, the protrusion 1138 may include a cylindrical member, and the complementary structure may include a ring-shaped member with a central cavity configured to receive a tip of the cylindrical member 138. The protrusion 1138 and the complementary structure may engage with a snap-fit arrangement. The user may be able to feel engagement between the protrusion 1138 and the complementary structure, via the actuation member 1068, when the actuation member 1068 reaches the intermediate position. The engagement may also help maintain the actuation member 1068 in the intermediate position, until the user separates the actuation member 1068 from the complementary structure. The protrusion 1138 and complementary structure may be used in embodiments where neither of the biasing members 1126 and 1132 is used, where one of the biasing members 1126 and 1132 is used, or where both of the biasing members 1126 and 1132 are used.

It is also contemplated that the helical needle 1044 may be received within outer housing or sheath 204 including a needle drive mechanism. For example, the outer housing or sheath 204 may include C-shaped distal opening 306, post 626 on its interior surface, and/or end wall 628 having helically shaped opening 630, for engaging the helical needle 1044 during its rotation, thereby driving the helical needle 1044 longitudinally. Additionally or alternatively, the outer housing or sheath 204 may include internal threaded region 622 engaged by external threaded region 624 on the outer surface of the shaft 1042, such that engagement between the threaded regions 622 and 624 during turning of the hypotube 1040 may drive the hypotube 1040 longitudinally in the same way a screw interacts with a nut.

Exemplary method steps associated with the above-described embodiments will now be described. A first step may include navigating the helical needle 1044 to a target area of a subject's body. This step may include inserting the helical needle 1044, shaft 1042, and outer sheath 1062 of the medical device 1010 into the subject's body.

It is contemplated that a catheter, endoscope, or similar insertion device (not shown) may be inserted into the subject's body and navigated to the target area. As known in the art, the insertion device may include a steering mechanism to help deflect distal portions of the insertion device, and help the insertion device navigate into and through the subject's body. For example, the insertion device may include a plurality of longitudinally extending cables or wires positioned around a circumference of the insertion device. The wires may be coupled to a proximal handle, including one or more knobs. Rotation of the one or more knobs may pull certain wires, and/or push certain wires, to steer/deflect the distal portion of the insertion device.

The insertion device may also include one or more lumens for receiving one or more instruments. For example, the insertion device may include a lumen for receiving an imaging device, such as a camera or sensor, allowing the imaging device to be positioned at or adjacent a distal end of the insertion device. The imaging device allows the user to visualize the target area. An exemplary insertion device (a video endoscope), is described in U.S. Pat. No. 7,578,786 B2 to Boulais et al., titled “Video Endoscope,” which was issued Aug. 25, 2009, and its contents are herein incorporated by reference. In some embodiments, the insertion device is a cystoscope, which may access the bladder via the urethra. In some embodiments, the insertion device is a laparoscope, which may access the bladder percutaneously or otherwise surgically, including but not limited to puncturing through the bladder wall. It is also contemplated that the insertion device may also include a lumen for another instrument. This other instrument may be configured to change a state of the injected substance. The other instrument may include, for example, a heated wire or laser, configured to crosslink or solidify the injected substance. Additionally or alternatively, the needle itself may be heated to bring about the change of state. Additionally or alternatively, fluids that solidify upon contact to form a new desired material, may be introduced via different lumens. Examples of such fluids are collagen and glutaraldehyde, and also cross-linked hydrogels. It is also contemplated that the needle may have multiple parallel lumens extending therethrough, for separating the fluids, and then allowing them to mix when exiting from the distal end of the needle.

The helical needle 1044, shaft 1042, and outer sheath 1062 may be inserted through a lumen of the insertion device after the insertion device has reached the target area, or may already be positioned within the insertion device during movement of the insertion device to the target area, such that deflection of the insertion device by the steering mechanism may also cause a similar deflection of the helical needle 1044, shaft 1042, and outer sheath 1062. A distal end of the needle 1044, shaft 1042, and/or outer sheath 1062 may be positioned at, adjacent to, or distally beyond the distal end of the insertion device. It is also contemplated that the distal end may be visualized by the imaging device. The housing 1012 of the medical device 1010 may remain outside of the subject's body, where it can be manipulated by the user.

The helical needle 1044 may be placed adjacent and/or against tissue at the target area. The target area may be the subject's bladder, and the tissue may be the tissue forming the bladder wall. Placing the needle adjacent and/or against the tissue may include abutting the tissue with the distal tip of the helical needle 1044. The user may accurately place the helical needle 1044 using the imaging device to visually guide the needle to the target area, and using the insertion device steering mechanism to position the helical needle 1044.

With the helical needle 1044 in position, penetration of the tissue with the helical needle 1044 may begin. Penetrating the tissue with the helical needle 1044 may include moving the actuation member 1068 toward the distal position from the intermediate position. As the actuation member 1068 is moved toward the distal position, the drive members 1078, 1080, 1082, 1084, and 1086, along with the bearing assembly 1116 and the pin vise 1118 gripping the hypotube 1040, may also move. These movements may cause rotation of the helical needle 1044 in a tissue penetrating direction.

As the actuation member 1068 is moved further toward the distal position, the helical needle 1044 may penetrate deeper into the tissue. When the needle has reached a desired depth, the user may stop moving the actuation member 1068. The desired depth may be the depth at which the distal opening 1050 at the distal end of the helical needle 1044 has reached a layer of tissue forming part of the bladder wall, or between layers forming part of the bladder wall.

With the helical needle 1044 at the desired depth, the user may deliver one or more substances into the hypotube 1040 using the syringe 1026. The substance may be injected in predetermined quantities. The substance may flow through the lumen 1046 of the hypotube 1040 and out the distal opening of the helical needle 1044 into the tissue.

After injecting the substance, the user may withdraw the helical needle 1044 from the tissue. Withdrawal may include moving the actuation member 1068 toward the intermediate position, and/or allowing the biasing member 1126 to move the actuation member 1068. As the actuation member 1068 is moved toward the intermediate position, the drive members 1078, 1080, 1082, 1084, and 1086, along with the bearing assembly 1116 and the pin vise 1118 gripping the hypotube 1040, may also move. These movements may cause rotation of the helical needle 1044 in a tissue disengaging direction. After the helical needle 1044 is withdrawn, the user may move, or allow the actuation member 1068 to move, back to the intermediate position.

When the actuation member 1068 has reached the intermediate position, a distal portion of the helical needle 1044 may remain in the tissue. To fully remove the helical needle 1044 from the tissue, the user may move the actuation member from the intermediate position toward the proximal position. As the actuation member 1068 is moved toward the proximal position, the drive members 1078, 1080, 1082, 1084, and 1086, along with the bearing assembly 1116 and the pin vise 1118 gripping the hypotube 1040, may also move. These movements may cause additional rotation of the helical needle 1044 in the tissue disengaging direction.

The user may reposition the helical needle 1044 adjacent and/or against tissue at another site in the target area. For example, the user may move the helical needle 1044, shaft 1042, and outer sheath 1062 using the steering mechanism of the insertion device. The user may use the imaging device to ensure the helical needle 1044 is moved to the desired site. Once there, the user may repeat the penetration, injection, and withdrawal steps listed above. It is contemplated that, for treatment of OAB, these steps may be repeated between 15 and 50 times to inject substances into 15 to 50 sites on the bladder wall. When the desired number of injections has been made, the medical device 1010 may be withdrawn from the subject's body.

Other embodiments of the present disclosure will be apparent to those skilled in the art from consideration of the specification and practice of the embodiments disclosed herein. It is intended that the specification and examples be considered as exemplary only, with a true scope and spirit of the disclosure being indicated by the following claims.

What is claimed is:

1. A medical device for injecting one or more substances, comprising:
a proximal handle assembly, including:
a housing,
a rotatable member rotatably coupled to the housing, and
a port configured to receive one or more substances;
an inner member coupled to the rotatable member and extending distal to the proximal handle assembly;
an outer member coupled to the housing, surrounding at least a portion of the inner member, and extending distal to the proximal handle assembly; and
a helical needle coupled to the inner member and extending distal to the inner member.

2. The medical device of claim 1, wherein a pathway for the one or more substances extends from the port to the helical needle.

3. The medical device of claim 2, wherein rotation of the rotatable member causes rotation of the inner member and the helical needle.

4. The medical device of claim 3, further including a needle drive mechanism configured to move the helical needle along at least one of the proximal and distal directions, when the rotatable member is rotated.

5. The medical device of claim 4, wherein the proximal handle assembly further includes:
a threaded shaft coupled to the rotatable member, and
a threaded recess in the housing, the threaded shaft and the threaded recess engaging to form at least part of the needle drive mechanism.

6. The medical device of claim 4, further including a needle housing coupled to the outer member, the needle housing extending distal to the outer member, wherein the housing is configured to receive at least a portion of the helical needle.

7. The medical device of claim 6, wherein the needle housing includes a distal surface with an opening, the distal surface of the needle housing and an outer surface of the helical needle engaging to form at least part of the needle drive mechanism.

8. The medical device of claim 6, wherein the needle housing includes an inner surface, and a post extending radially inwardly from the inner surface, the post and an outer surface of the helical needle engaging to form at least part of the needle drive mechanism.

9. The medical device of claim 4, wherein the outer member includes a distal surface with an opening, an outer surface of the helical needle and the distal surface of the outer member engaging to form at least part of the needle drive mechanism.

10. The medical device of claim 4, further including a male threaded coupler on the inner member, and a female threaded portion on the outer member, wherein the male threaded coupler engages the female threaded portion, and the male threaded coupler and the female threaded portion to form at least part of the needle drive mechanism.

11. The medical device of claim 4, wherein the outer member includes an inner surface, and a post extending radially inwardly from the inner surface, the post and an outer surface of the helical needle engaging to form at least part of the needle drive mechanism.

12. The medical device of claim 4, wherein the outer member includes a distal end wall, and a helical passage through the end wall configured to receive at least a portion of the helical needle, the helical passage and an outer surface of the helical needle engaging to form at least a portion of the needle drive mechanism.

13. The medical device of claim 4, wherein the inner member includes a substantially flat distal end face, and a proximal end of the helical needle is coupled to the distal end face of the inner member, adjacent an edge of the distal end face of the inner member.

14. The medical device of claim 1, further comprising an insertion device including:
a lumen configured to receive the inner member, outer member, and helical needle, and
a steering mechanism configured to deflect a distal portion of the insertion device.

15. A medical device for injecting a substance, comprising:
a rotatable tube coupled to a distal helical needle;
a drive assembly coupled to the rotatable tube, wherein the drive assembly is configured to rotate the rotatable tube;
an actuation member coupled to the drive assembly, wherein the actuation member is configured to move between a proximal position and a distal position, to actuate the drive assembly to rotate the rotatable tube; and
a connector on the rotatable tube configured to receive a source of the substance.

16. The medical device of claim 15, wherein the drive assembly is configured to convert planar movement of the actuation member between the proximal position and the distal position, into rotation of the rotatable tube about a longitudinal axis of the rotatable tube.

17. The medical device of claim 16, wherein the drive assembly includes a plurality of gears.

18. The medical device of claim 17, wherein the plurality of gears include a first gear coupled to the actuation member, configured to rotate about an axis substantially perpendicular to the longitudinal axis of the rotatable tube, and a second gear coupled to the rotatable tube, and configured to rotate about the longitudinal axis of the rotatable tube.

19. The medical device of claim 15, further including at least one biasing member coupled to the actuation member, the at least one biasing member being configured to position the actuation member in an intermediate position between the proximal position and the distal position.

20. A method for injecting a substance into tissue with a medical device, the method comprising:
engaging a surface of the tissue with a helical needle of the medical device;
rotating the helical needle in a tissue penetrating direction to penetrate the tissue, wherein rotating the helical needle in the tissue penetrating direction includes moving an actuation member from a rest position to a first position, to rotate the helical needle through a first angle of rotation in the tissue penetrating direction;
injecting the substance into the tissue through the helical needle; and
rotating the helical needle in a disengaging direction opposite the tissue penetrating direction to withdraw the helical needle from the tissue.
